(19) United States
(12) Patent Application Publication
(10) Pub. No.: US 2014/0066833 A1
(43) Pub. Date: Mar. 6, 2014

(54) EXPANDABLE FLUID DRAINAGE IMPLANTS AND ASSOCIATED DELIVERY DEVICES AND METHODS

(71) Applicant: CLEARLIX LTD., Rehovot, IL (US)

(72) Inventors: Ira YARON, Har Adar (IL); Yonatan BEN-ZVI, Kiryat Tiv'on (IL)

(21) Appl. No.: 13/973,628

(22) Filed: Aug. 22, 2013

Related U.S. Application Data
(60) Provisional application No. 61/693,896, filed on Aug. 28, 2012.

(51) Int. Cl. A61F 9/007

(52) U.S. Cl. CPC A61F 9/00781
USPC 604/9

(57) ABSTRACT

A drainage implant is provided for the drainage of aqueous humor. The implant may comprise a collector, a connector and disperser. The collector and/or disperser may be self-expandable and can be held in an unexpanded condition by a delivery device. When positioned for implantation, the collector and disperser are ejected from the delivery device, whereby upon being ejected from the delivery device they can expand to a pre-defined final shape in the desired place. A delivery device for implanting the implant may have a shaft for accommodating the implant and a cutting edge that may be used to create a pocket or reservoir in the tissue where the implant device is to be implanted. Methods of implanting an implant are also disclosed.

### CROSS REFERENCE TO RELATED APPLICATIONS

The present application claims priority to U.S. provisional application Ser. No. 61/693,896 filed Aug. 28, 2012, the disclosure of which is incorporated herein by reference in its entirety.

### FIELD OF THE INVENTION

The invention relates to devices for the treatment of glaucoma, in particular to drainage implants that can be implanted in the eye to allow the drainage of aqueous humor in order to help regulate intraocular pressure (IOP). The invention also relates to associated delivery devices and methods.

### BACKGROUND OF THE INVENTION

Glaucoma is a disease caused by an increased IOP due to the diseased eye having a deficiency in the drainage of aqueous humor. In a normal eye, aqueous humor is continuously made in the eye and flows out of the eye through known channels. In some cases, the normal flow of aqueous humor from the eye can become slowed or blocked, leading to increased IOP, and possibly to glaucoma.

Devices have been proposed previously to facilitate the flow of aqueous humor from the eye. For example, U.S. Pat. No. 4,968,296 to Ritch disclosed one such proposal. The disclosure of that patent is hereby incorporated herein by reference.

The suitability and success of a drainage implant depend on a variety of factors, for example the ease of implantation, the time required for implantation, the invasiveness of the implantation procedure, the potential for complications during or after the implantation procedure, the potential for infections, the expected recovery time after the implantation procedure, the susceptibility of the procedure to cause the generation of scar tissue (which could cause blockage and inhibit proper fluid flow), the performance of the implanted device in regulating fluid flow, the ability of the device to stay in the proper position once implanted, general safety, performance, cost, overall clinical outcome and other factors. There is a continuing need for improvements in glaucoma treatment.

### SUMMARY OF THE INVENTION

The present invention provides devices for the treatment of glaucoma, in particular drainage implants that can be implanted in the eye to allow the drainage of aqueous humor. The invention also provides associated delivery devices and methods.

In some embodiments, the implant device comprises a collector, a connector and a disperser. In one example embodiment, the collector and disperser are expandable and have the ability to be held in a low-profile unexpanded condition inside a lumen of, or on a shaft of, a delivery device. When positioned for implantation, the collector and disperser are ejected from the delivery device, whereby upon being ejected from the delivery device they expand to a pre-defined final shape in the desired place. In another example embodiment, one of the collector or the disperser is expandable.

In some embodiments, a delivery device for implanting an implant device has a shaft, which may have a lumen, and a cutting edge. The cutting edge may be used to create a pocket or reservoir in the tissue where the implant device is to be implanted. The shaft may accommodate the implant device. For example, the implant may be positioned inside a lumen of the shaft, or the implant may be positioned on the outside of the shaft.

In some embodiments, a method of implanting an implant device may be accomplished using a delivery device having a shaft that accommodates the implant device, either inside a lumen of the shaft or on the outside of the shaft. The implant device has an expandable collector and/or disperser, which is held during delivery of the implant device in a low-profile unexpanded condition inside the lumen of, or on the shaft of, the delivery device. In the implantation method, the delivery device is positioned at the desired site, whereupon the implant device is ejected from the delivery device. When the expandable collector and/or disperser is ejected from the delivery device, it expands to a pre-defined final shape in the desired place. In some embodiments, an ab-interno implantation method is used to implant the device. In other embodiments, an ab-externo implantation method is used to implant the device. Both the ab-interno and the ab-externo implantation methods may be blebless or bleb-forming surgery.

When the implant device is implanted, the implant device allows uninterrupted flow of fluid from one side to the other side of the device. For example, when the collector is positioned in the anterior chamber on the inside of the sclera in the area of the anterior chamber angle, and the disperser is positioned in a pocket formed within the sclera, the implant device allows fluid flow from the anterior chamber to a reservoir formed by the pocket.

In certain embodiments, the expandable collector and/or disperser may have a generally spiral or conical-helical shape. In alternative embodiments, the expandable collector and/or disperser may have another expandable shape, such as an expandable fish-tail shape, an expandable set of fingers, one or more expandable side arms, an expandable frame, an expandable dish, or another suitable expandable shape. The expandable collector and/or disperser may be held in an unexpanded condition by a delivery device, for example inside the lumen of a delivery device or on the outside of the shaft of a delivery device. When positioned for implantation, the expandable collector and/or disperser is ejected from the delivery device, whereby upon being ejected from the delivery device it expands to a pre-defined final shape in the desired place.

### BRIEF DESCRIPTION OF THE DRAWINGS

FIG. 1A shows a top view of an implant device comprising an expandable spiral collector and an expandable conical helix disperser.

FIG. 1B shows a side view of the implant device of FIG. 1A.

FIG. 2 shows a schematic view of another implant device comprising an expandable spiral collector and an expandable conical helix disperser.

FIG. 3 shows a schematic view of the implant device of FIG. 2 implanted in an eye.

FIG. 4A shows a delivery device for implanting an implant device.

FIG. 4B shows a view of a hollow shaft of the delivery device of FIG. 4A, with an implant device positioned inside the lumen of the hollow shaft.

FIG. 4C shows another embodiment of a delivery device for implanting an implant device.

FIGS. 5A-5G show steps in an ab-interno implantation procedure. FIG. 5A shows the delivery device being passed across the anterior chamber. FIG. 5B shows a cutting edge of the delivery device advanced into the sub-scleral space. FIG. 5C shows the cutting edge being used to form a pocket in the sclera. FIG. 5D shows the disperser being ejected from the lumen of the delivery device, whereupon it expands into the pocket in the sclera. FIG. 5E shows the delivery device being withdrawn until the distal end of the lumen is withdrawn back into the anterior chamber. FIG. 5F shows the collector being ejected from the lumen of the delivery device, whereupon it expands to anchor the implant device. FIG. 5G shows the implant in place after the delivery device has been withdrawn.

FIG. 5H shows an alternative use of a cutting edge of the delivery system (compared to that shown in FIG. 5B)—advancing beyond the sub-scleral space up to the sub-conjunctival space. FIG. 5I shows a side view of the FIG. 5H cutting edge path, from the anterior chamber up to the sub-conjunctival space.

FIG. 6 shows an implant device comprising an expandable fish-tail collector and an expandable spiral disperser.

FIG. 7 shows an implant device comprising an expandable three-dimensional fish-tail collector and a disperser in the form of an expandable set of fingers.

FIGS. 8A-8D show an implant device comprising a collector with expandable side arms and a disperser with expandable side arms. FIG. 8A shows a top view, FIG. 8B shows an end view, and FIG. 8C shows a side view of the implant device in the constrained configuration. FIG. 8D shows a side view of the implant device in the unconstrained, expanded configuration.

FIG. 8E shows an implant device comprising a collector with expandable side arms as in FIGS. 8A-8D and a disperser similar to that in FIGS. 1A-1B.

FIG. 9A shows an implant device comprising a collector with an expandable frame and a disperser with an expandable frame.

FIG. 9B shows the implant of FIG. 9A loaded inside a delivery device.

FIG. 10 shows an implant device comprising a disperser with an expandable frame.

FIG. 11 shows an implant device comprising an expandable fish-tail collector and a disperser in the form of an expandable set of fingers.

### DETAILED DESCRIPTION

Certain embodiments of devices and methods of using them are described herein with reference to the accompanying drawings. These embodiments are only examples, as numerous variations of the invention disclosed herein are possible within the scope of the appended claims.

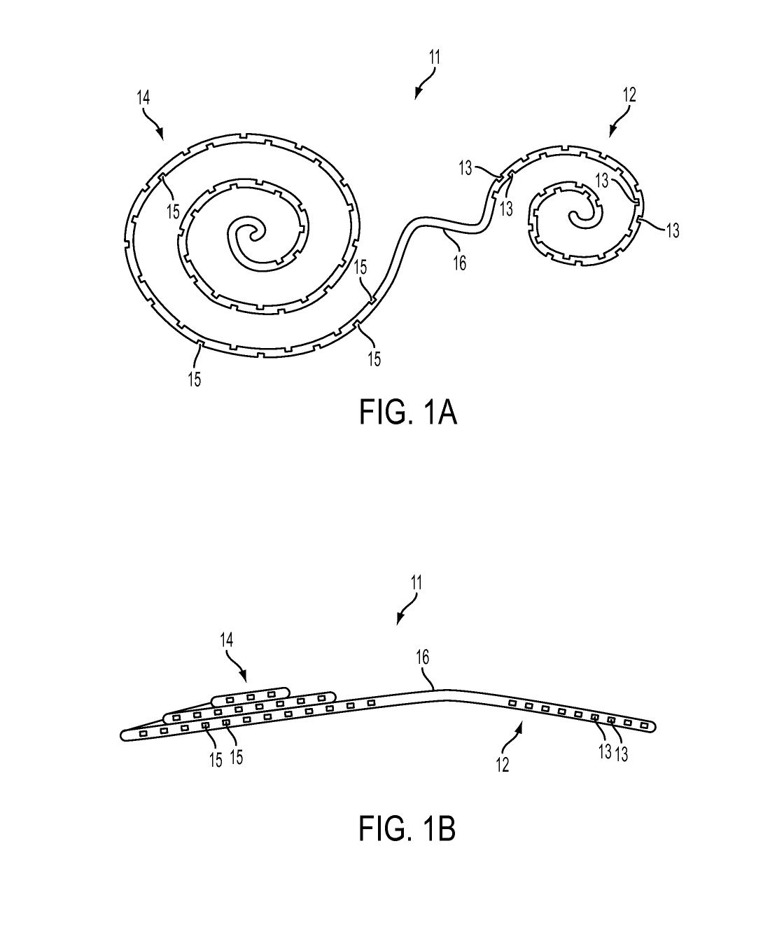

FIG. 1A shows a top view of an implant device 11, and FIG. 1B shows a side view of the same implant device 11. The implant device 11 is formed of a tube made of a suitable flexible material that allows the implant device to be compressed to a low profile in a constrained condition and that allows the implant device to self-expand to an expanded profile when unconstrained. For example, the material may be a shape memory material, such as nitinol or another suitable shape memory alloy, or another suitable flexible material such as a suitable plastic or other metal. The cross-section of the tube may be circular, elliptical, rectangular or any other suitable shape.

In the unconstrained or “remembered” or expanded shape, the implant device 11 has the shape shown in FIGS. 1A and 1B. As shown in these figures, the implant device 11 comprises a spiral collector 12 and a conical helix disperser 14 connected by a connector 16.

As can be seen in FIGS. 1A and 1B, the collector 12 has holes or slits 13 in the wall of the tube. These holes or slits 13 allow fluid to enter the tube at the collector 12, together with the tube's natural hollow profile.

The connector 16 is formed of the portion of the tube of the implant device 11 between the collector 12 and the disperser 14. In the embodiment shown in FIGS. 1A and 1B, there are no holes or slits in the connector 16. In alternative embodiments, one or more holes or slits may be provided in the connector 16. When the connector 16 is positioned in the Schlemm's Canal (as described below), the provision of one or more holes or slits can allow the drainage of aqueous humor directly into the Schlemm's Canal.

The expanded disperser 14 has a conical helix shape that can be seen in FIGS. 1A and 1B. The disperser 14 has holes or slits 15 in the wall of the tube. These holes or slits 15 allow fluid to exit the tube at the disperser 14, together with the tube's natural hollow profile.

The implant device 11 of FIGS. 1A and 1B may be implanted in an eye to facilitate drainage of aqueous humor in order to regulate IOP. In one example positioning, the implant device 11 is implanted at the area of the anterior chamber angle. When implanted, the collector 12 is placed in the anterior chamber against the inside surface of the sclera, near the Schlemm's Canal, the disperser 14 is placed in a pocket formed in the sclera (as described below), and the connector 16 extends between and connects the collector 12 and the disperser 14. When the implant device 11 is implanted, the fluid flows from the high pressure area in the anterior chamber to the reservoir in the sclera, which has a lower pressure. The fluid enters the tube of the implant device 11 through the holes or slits 13 in the collector 12. The fluid flows from the collector 12 through the connector 16 to the disperser 14. The fluid then exits the tube of the implant device 11 through the holes or slits 15 in the disperser 14. The wide shape of the disperser 14 directs the fluid widely within the pocket.

The spiral or helical shape of the collector and disperser acts as an anchor and reduces the ability of the device to migrate. The collector 12 anchors the device 11 to prevent it from moving into the sclera. The disperser 14 anchors the device to prevent it from moving into the anterior chamber. The height, width and depth of the disperser 14 keep the walls of the pocket away from each other, ensuring long-lasting functioning of the reservoir.

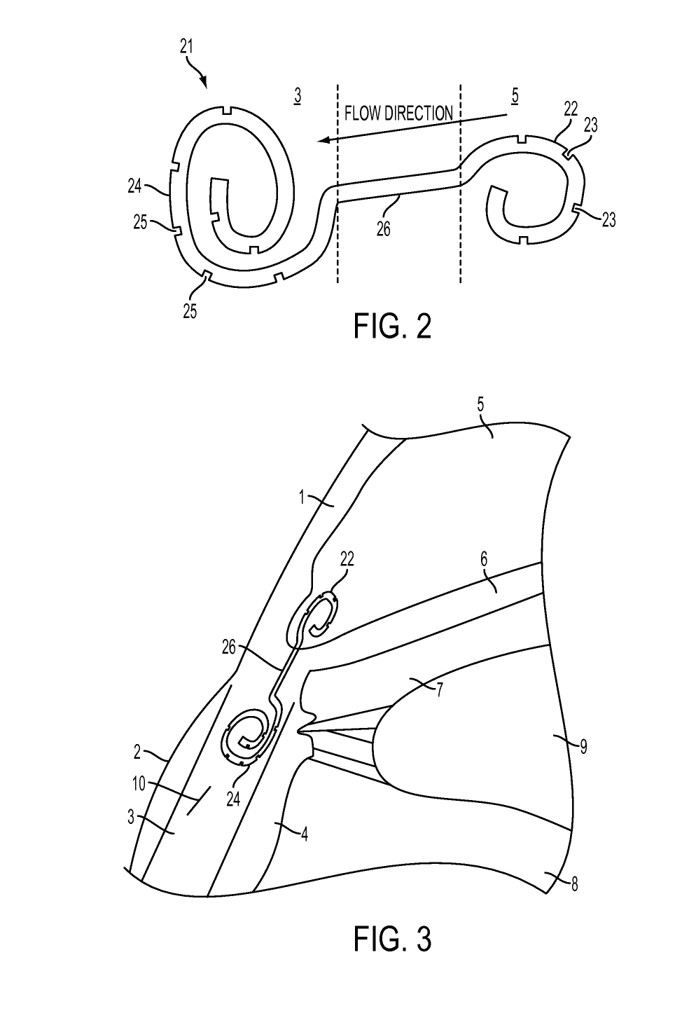

FIG. 2 shows a schematic view of another implant device 21 comprising an expandable spiral collector 22 and an expandable conical helix disperser 24. Like the implant device 11, the implant device 21 may be formed of a tube made of a suitable flexible material as described above. The cross-section of the tube may be circular, elliptical or any other suitable shape. FIG. 2 shows the implant device 21 in the expanded or unconstrained (“remembered”) shape. As with the implant device 11, the collector 22 and the disperser 24 have holes or slits 23, 25 in the wall of the tube. Holes or slits may also be provided in the connector 26.

FIG. 3 illustrates an example implantation location for an implant device. The implant device 11, the implant device 21, and the other implant devices described and illustrated herein may be implanted in the same location as that illustrated in FIG. 3. The eye includes the cornea 1, conjunctiva 2, sclera 3, ciliary muscle 4, anterior chamber 5, iris 6, posterior chamber 7, vitreous 8, lens 9, and collector vessels/channels 10. In this example positioning, the implant device 21 is shown as an example. The implant device is implanted at the area of the anterior chamber angle. When implanted, the collector 22 is placed in the anterior chamber 5 against the inside surface of the sclera 3, near the Schlemm's Canal, the disperser 24 is placed in a pocket formed in the sclera 3, and the connector 26 extends between and connects the collector 22 and the disperser 24. When the implant device is implanted, the fluid flows from the high pressure area in the anterior chamber 5 to the reservoir in the sclera 3, which has a lower pressure. The fluid enters the tube of the implant device 21 through the holes or slits 23 in the collector 22. The fluid flows from the collector 22 through the connector 26 to the disperser 24. The fluid then exits the tube of the implant device 21 through the holes or slits 25 in the disperser 24. The wide shape of the disperser 24 directs the fluid widely within the pocket.

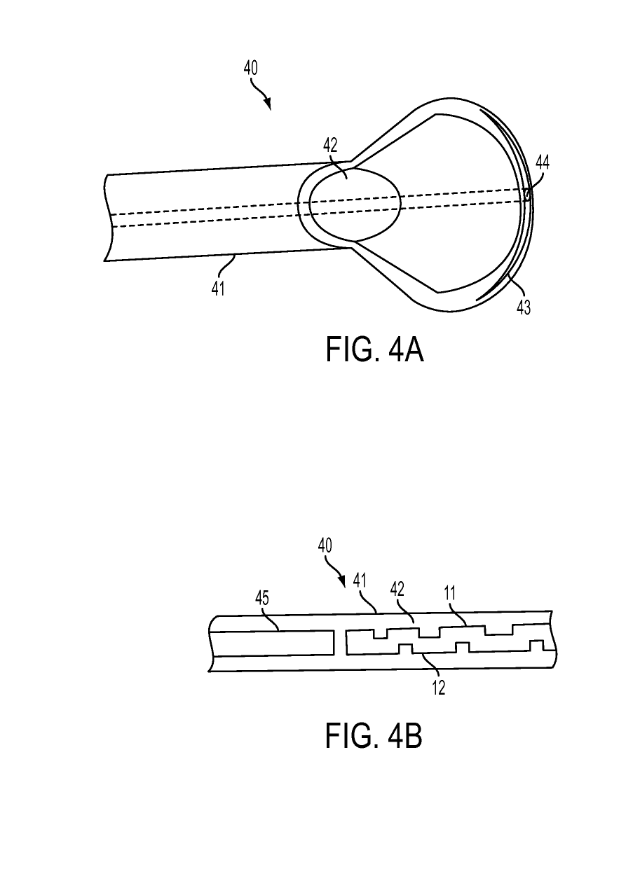

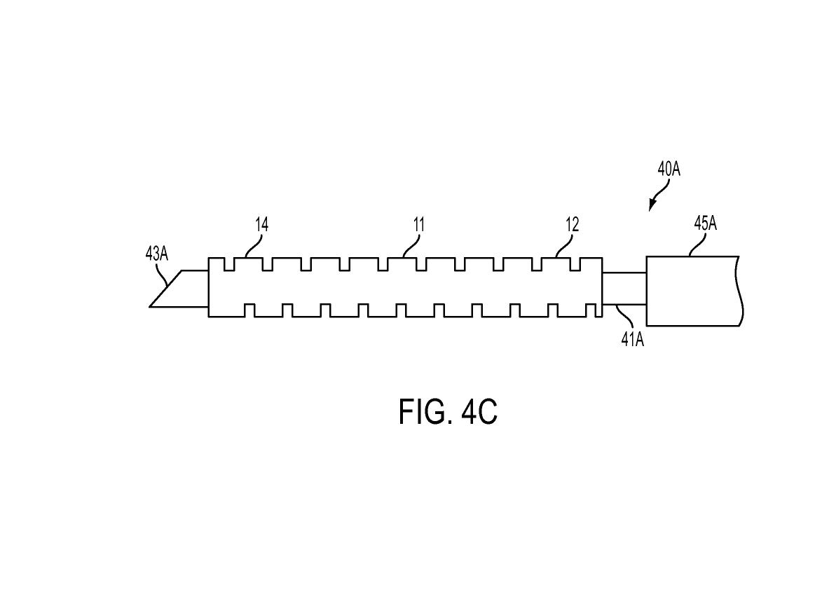

FIG. 4A shows a delivery device 40 for implanting an implant device as described and illustrated herein. The delivery device 40 comprises a hollow shaft 41 having an internal lumen 42, a sharp cutting edge 43 at the distal end of the hollow shaft 41, and an illuminator 44, such as a fiber optic tip. FIG. 4B shows a view inside a part of the hollow shaft 41 of the delivery device 40 of FIG. 4A, with an implant device 11 positioned inside the lumen 42 of the hollow shaft 41. The implant device 11 is positioned inside the lumen 42 distal to (i.e., closer to the outlet than) an injector 45, which is also positioned inside the lumen 42 of the hollow shaft 41. In addition to being used for retaining the implant device during the implantation procedure, the hollow shaft 41 may be used for irrigation, aspiration and/or delivery of viscoelastic material (as described below). Additional lumens may be provided for these or other purposes. The cutting edge 43 may be provided, for example, by a crescent blade at the distal end of the delivery device 40. More than one illuminator 44 or fiber optic may be used, which can improve visualization and orientation. The delivery device 40 may include a pressure probe for monitoring the IOP in real time.

As can be appreciated from FIG. 4B, when the implant device 11 (or another similar implant device as described herein) is loaded into the lumen 42 of the delivery device 40, the implant device 11 is held by the delivery device 40 in a low-profile, constrained condition. For example, when loaded in a lumen 42 of a delivery device 40, the implant device 11 is straightened into a substantially straight shape. In the example of FIG. 4B, the portion of the implant 11 that is adjacent the distal end of the injector 45 is the substantially straightened collector 12. Because of its shape memory characteristics, the implant can be temporarily deformed into a low-profile shape for loading into the delivery device and for delivery to the implantation site. The shaft of the delivery device holds and maintains the implant in its low-profile configuration during delivery of the implant. When the implant is ejected from the delivery device, it self-expands to its unconstrained, relaxed or “remembered” shape.

FIG. 4C shows another embodiment of a delivery device for implanting an implant device as described and illustrated herein. FIG. 4C shows a delivery device 40A comprising a shaft 41A and a sharp cutting edge 43A at the distal end of the shaft 41A. As shown in FIG. 4C, an implant device 11 is positioned on the outside of the shaft 41A, with the shaft 41A passing through the internal lumen of the implant device 11. The implant device 11 is positioned on the shaft 41A distal to (i.e., closer to the ejection end than) an injector 45A, which is also positioned on the outside of the shaft 41A.

As can be appreciated from FIG. 4C, when the implant device 11 (or another similar implant device as described herein) is loaded onto the shaft 41A of the delivery device 40A, the implant device 11 is held and maintained by the delivery device 40A in a constrained, low-profile condition. For example, when loaded onto the shaft 41A of a delivery device 40A, the implant device 11 is straightened into a substantially straight shape, and held and maintained in that position by the shaft 41A passing through the internal lumen of the implant device. The shaft 41A is more rigid than the implant device 11, and the shaft 41A is sufficiently rigid to maintain the implant device 11 in its constrained low-profile configuration. In the example of FIG. 4C, the portion of the implant 11 that is adjacent the distal end of the injector 45A is the substantially straightened collector 12. Because of its shape memory characteristics, the implant can be temporarily deformed into a low-profile shape for loading onto the delivery device and for delivery to the implantation site. The shaft of the delivery device holds and maintains the implant in its low-profile configuration during delivery of the implant. When the implant is ejected from the delivery device, by the injector 45A being moved distally on the shaft 41A and/or by the shaft 41A being withdrawn proximally relative to the injector 45A, the implant self-expands to its unconstrained, relaxed or “remembered” shape.

In addition to being used for retaining the implant device during the implantation procedure, the shaft 41A may be hollow and may be used for irrigation, aspiration and/or delivery of viscoelastic material. Additional lumens may be provided for these or other purposes. The cutting edge 43A may be provided, for example, by a crescent blade at the distal end of the delivery device 40A. An illuminator may be used, similar to illuminator 44. The delivery device 40A may include a pressure probe for monitoring the IOP in real time.

As can be appreciated, the slits or other openings in the expandable portion(s) of the implant can increase the flexibility of the implant so that it can be better accommodated in or on the shaft of the delivery device. The slits or openings may be formed and positioned to assist in flexibility. A single slit or opening may be used, such as a single helical slit along the whole tube.

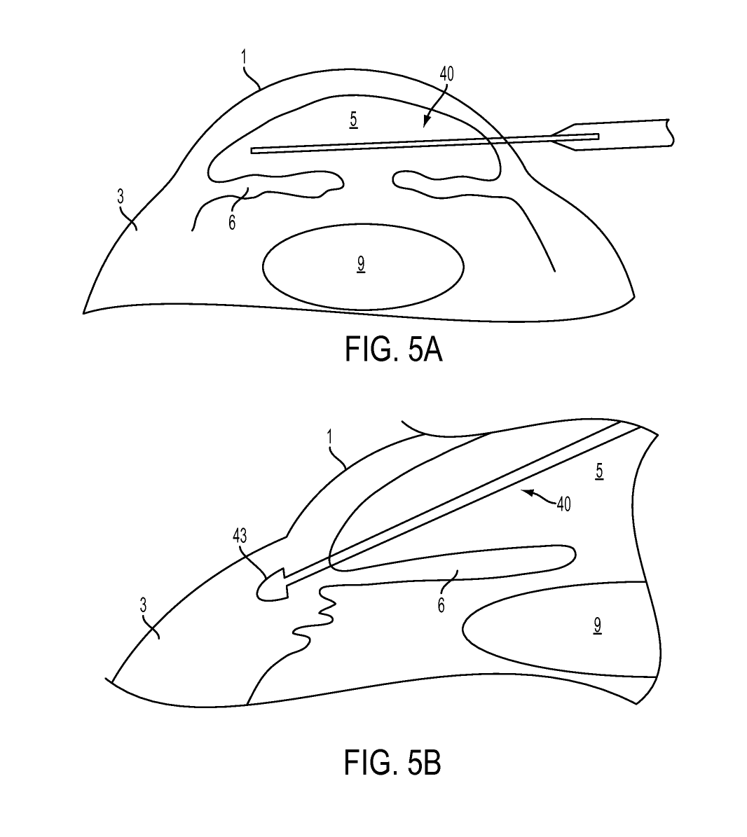

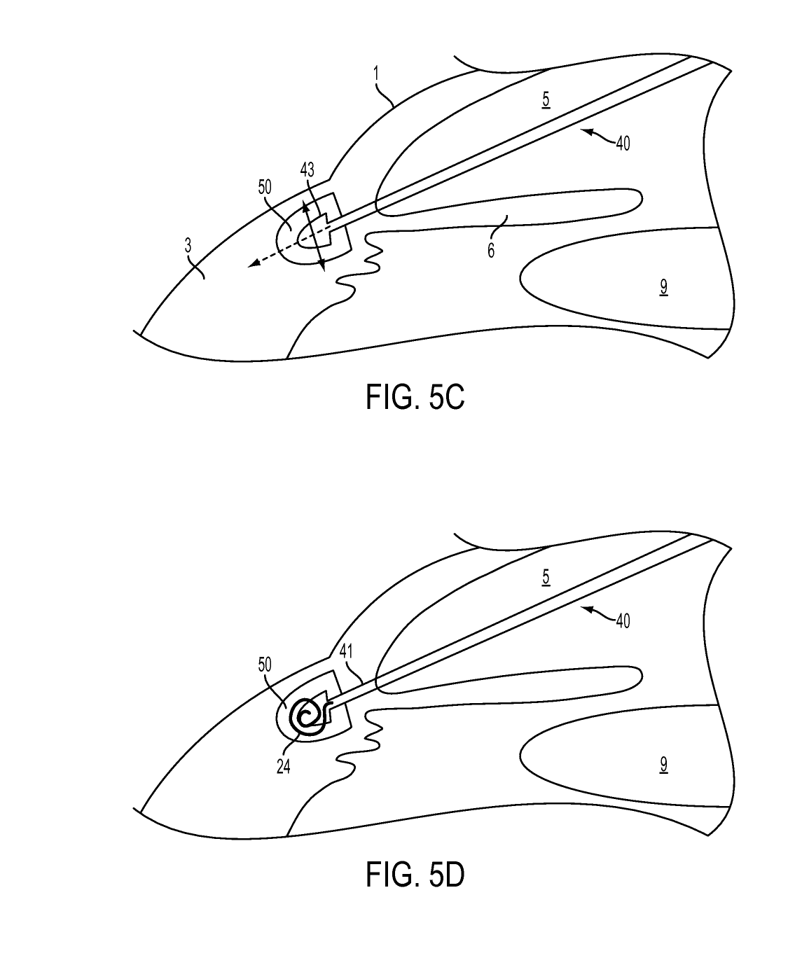

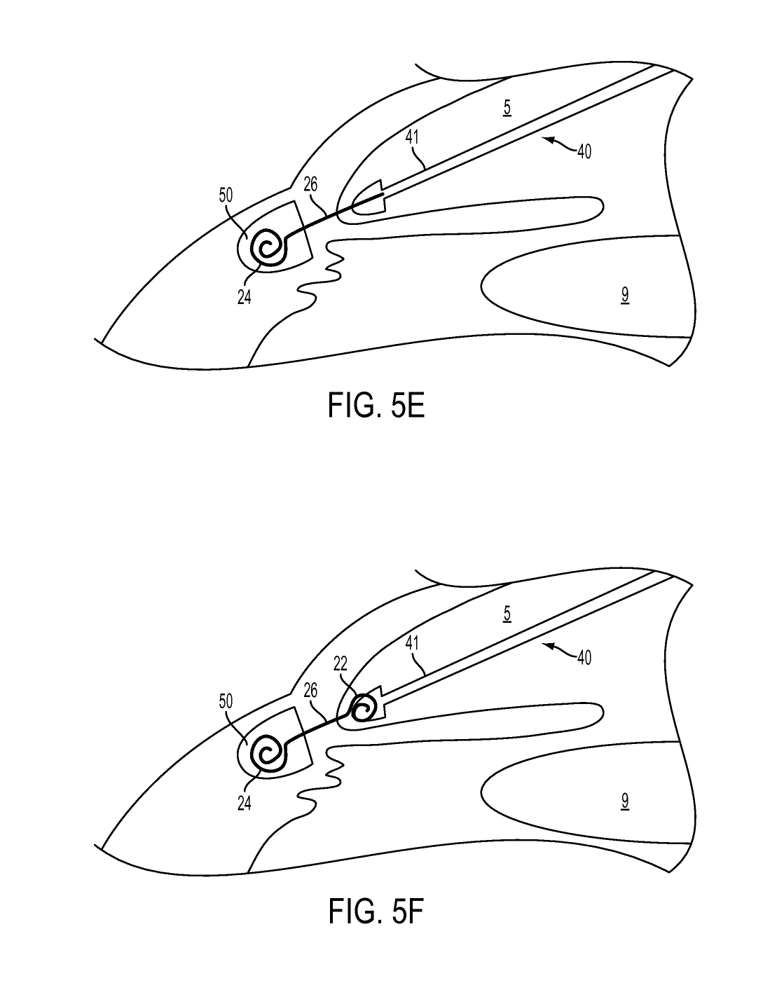

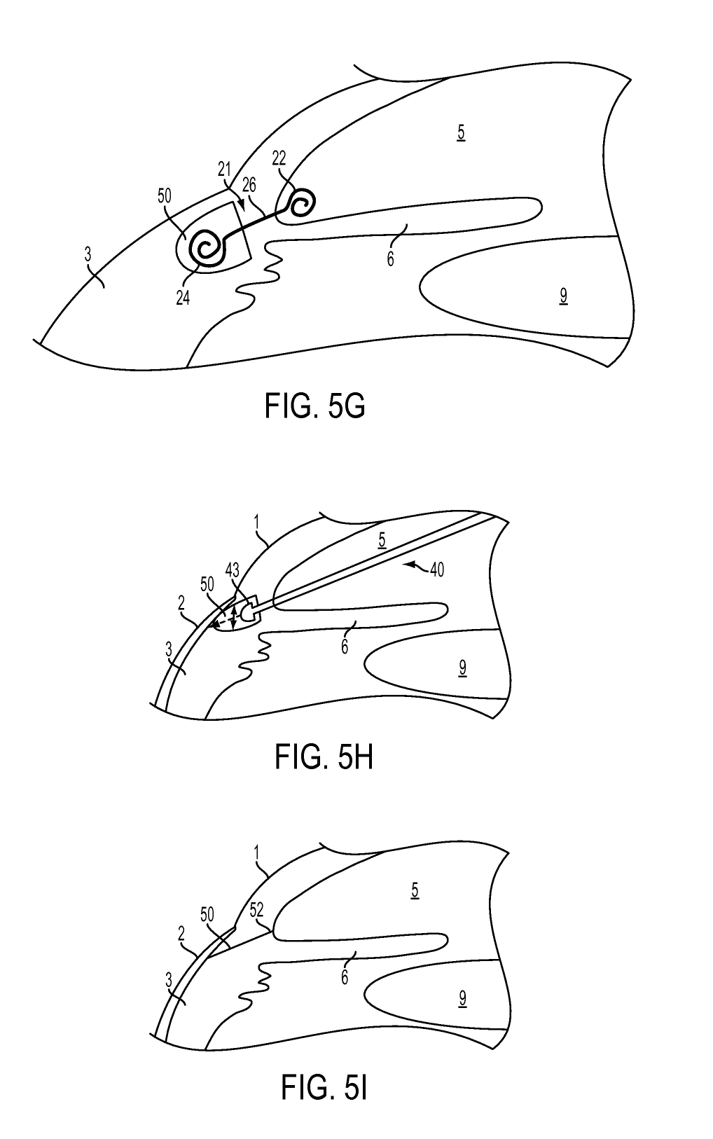

FIGS. 5A-5G show steps in an ab-interno implantation procedure, using a delivery device like the delivery device 40 shown in FIGS. 4A-B. First, the implant is loaded into the lumen of the delivery device 40 so that it is held in its constrained position. In this way, the expandable portion(s) of the implant can be delivered to the appropriate position with minimal tissue disruption. If a delivery device like the delivery device 40A shown in FIG. 4C is used, the implant is loaded onto the shaft 41A of the delivery device 40A so that it is held in its constrained position, thereby similarly allowing the expandable portion(s) of the implant to be delivered to the appropriate position with minimal tissue disruption. Then, as is known in the art for ab-interno procedures, the delivery device 40 or 40A is advanced through an incision in the cornea 1 into the anterior chamber 5 at a position opposite the intended implantation site. Then, as shown in FIG. 5A, the delivery device 40 or 40A is passed across the anterior chamber 5 to the angle between the cornea 1 and the iris 6.

At the intended implantation site, as shown in FIG. 5B, the cutting edge 43 or 43A of the delivery device 40 or 40A is advanced into the tissue. The cutting edge 43 or 43A is advanced into a sub-scleral space within the sclera 3. The cutting edge 43 or 43A may first penetrate the tissue in the Schlemm's Canal and then further into the sub-scleral space. Because the shaft 41 or 41A of the delivery device 40 or 40A has a low profile, and because the implant device is held at a low profile by the delivery device 40 or 40A when passing from the anterior chamber to the desired position, tissue disruption is minimized.

When the cutting edge 43 or 43A reaches the desired position in the sub-scleral space, the cutting edge 43 or 43A may be used, as shown in FIG. 5C, to form a pocket 50 within the sclera 3. The cutting edge 43 or 43A allows the physician to create a wide and long pocket 50 in the sclera 3 that will later serve as a reservoir. The illuminator 44 helps the physician to see where the cutting edge 43 or 43A is located and to make an accurate cut. The illuminated tip of the cutting edge 43 or 43A is visible through the conjunctiva 2 and the sclera 3.

The pocket 50 may be formed by moving the cutting edge 43 or 43A generally parallel to the sclera until desired shape and size is achieved. The cutting edge 43 or 43A may be advanced in different directions, as represented by the arrows in FIG. 5C. In one example of forming a pocket 50, a 1 mm to 2 mm outside diameter crescent sharp edge 43 or 43A may be moved to create a pocket that is 2 mm to 4 mm wide and 2 mm to 4 mm long. In another example, a pocket is created that is 6 mm wide and 6 mm long. Other dimensions are of course possible.

Through the hollow shaft 41 or 41A (if hollow), it is possible to inject viscoelastic material in order to keep the pocket 50 formed and to reduce the flow of aqueous humor following the procedure. This may reduce the risk of overflow post-operation. In addition, saline may be injected through the hollow shaft to elevate the roof of the pocket 50 during the implantation process. Thus, an intra-scleral bleb may be formed. In addition, aspiration of tissue particles cut away by the cutting edge 43 or 43A or aspiration of fluids may be carried out through the hollow shaft. The hollow shaft also may be used for irrigation purposes.

Once the pocket 50 is formed, the disperser 24 is ejected into the pocket 50. This is performed by holding the position of the hollow shaft 41 of the delivery device 40 while advancing the injector 45 within the hollow shaft 41. If the delivery device 40A is used, this is performed by holding the position of the shaft 41A of the delivery device 40A while advancing the injector 45A on the outside of the shaft 41A. The injector 45 or 45A pushes against the implant device, ejecting the portion of the implant device loaded toward the distal-most end of the lumen 42 or shaft 41A. In this embodiment, the implant device is loaded with the disperser 24 toward the distal-most end of the lumen 42 or shaft 41A and with the collector 22 toward the injector 45 or 45A. At this stage, the injector 45 or 45A is advanced only far enough to eject the disperser 24 portion of the implant device. When the disperser 24 is ejected from the lumen 42 or shaft 41A of the delivery device 40 or 40A, it self-expands to its unconstrained, pre-defined shape within the pocket 50. If desired, the unconstrained disperser may be slightly larger than the created pocket, thereby creating some constant pocket-stretching force. FIG. 5D shows the disperser 24 being ejected from the lumen 42 of the shaft 41 of the delivery device 40, whereupon the disperser 24 expands into its pre-defined final shape within the pocket 50 in the sclera 3.

Once the disperser 24 is in place in the pocket 50, the delivery device 40 is withdrawn. As it is being withdrawn, the injector 45 is used to eject the connector portion 26 of the implant device from the lumen 42 of the shaft 41 of the delivery device 40. This is shown in FIG. 5E. If the delivery device 40A is used, as the shaft 41A is withdrawn, the injector 45A is used to eject the connector portion 26 of the implant device from the outside of the shaft 41A of the delivery device 40A.

Once the delivery device 40 or 40A has been withdrawn to the point that the distal end of the lumen 42 of the shaft 41 or the distal end of the shaft 41A is in the anterior chamber 5, as shown in FIG. 5E, the injector 45 or 45A is used to eject the collector 22. As before, in the case of the delivery device 40, this is performed by holding the position of the hollow shaft 41 of the delivery device 40 while advancing the injector 45 within the hollow shaft 41. In the case of the delivery device 40A, this is performed by holding the position of the shaft 41A of the delivery device 40A while advancing the injector 45A over the shaft 41A. The injector 45 or 45A pushes against the implant device, ejecting the collector 22, whereupon the collector 22 expands to its pre-defined final shape in the desired place. FIG. 5F shows the collector 22 being ejected from the lumen 42 of the delivery device 40, whereupon it expands in the anterior chamber 5 to anchor the implant device.

Once the implant is in place, the delivery device 40 or 40A is withdrawn from the eye. FIG. 5G shows the implant 21 in place after the delivery device 40 or 40A has been withdrawn. In can be appreciated that in this ab-interno procedure, at the area of the implantation, the conjunctiva 2 remains intact. In such a procedure where the pocket 50 is formed beneath the outer surface of the sclera 3, the outer surface of the sclera 3 also remains intact. The ability to keep the conjunctiva 2 and in certain embodiments also the outer surface of the sclera 3 intact can help achieve a positive clinical outcome.

Alternatives to the above-described procedure are possible. In one alternative, the pocket 50 is formed to connect to the subconjunctival space between the conjunctiva 2 and the sclera 3. The pocket 50 may be formed to reach the subconjunctival space, or one or more channels may be formed connecting the pocket 50 to the subconjunctival space. This facilitates the flow of aqueous humor from the implant device into the subconjunctival space. This may be utilized, for example, when an additional filtering mechanism is needed. As one example, it is possible to facilitate flow into the sub-conjunctival space by performing the following steps. First, saline, lidocaine, viscoelastic material, or another suitable material is injected between the sclera 3 and the conjunctiva 2, which results in elevating the tissue of the conjunctiva 2 away from the sclera 3, thereby forming a space between the sclera 3 and the conjunctiva 2. This reduces the risk of harming the conjunctiva 2 during subsequent cutting of the pocket 50. After the conjunctiva 2 is raised, a connection is made between the pocket 50 and the subconjunctival space. This may be done, for example, using the cutting edge 43 or 43A.

FIG. 5H shows this alternative, in which the cutting edge 43 or 43A is used to form the pocket 50 near the outer surface of the sclera 3, and the cutting edge 43 or 43A is used to cut a connection channel through the sclera 3 to the subconjunctival space, thereby connecting the pocket 50 with the space between the sclera 3 and the conjunctiva 2. Thus, aqueous humor can flow through the implant device from the anterior chamber 5 to the sub-conjunctival filtering space. In one example, the disperser of the implant device is implanted to lie completely within the sclera 3, permitting flow from there to the subconjunctival space. In other examples, the disperser of the implant device is implanted to lie only partially within the sclera 3, or in an area between the conjunctiva 2 and the sclera 3, such that the disperser can output the aqueous humor, in whole or in part, directly into the subconjunctival space.

It will be appreciated that the views shown herein are schematic representations and that, for example, the pocket 50 may take various forms. For example, the pocket 50 may be cut to have its largest dimensions generally parallel to the sclera 3 or only at a slight angle to the sclera 3. FIG. 5I shows a line 52 representing an example of a direction in which the cutting edge 43 is directed through the sclera 3. The pocket 50 may be formed along this line 52.

In another alternative, an ab-externo implantation method is used. In an example of an ab-externo method, the physician first cuts the conjunctiva, then forms the intra-scleral pocket. In this procedure, the pocket may be formed in a similar manner as current deep sclerectomy procedures. Alternatively, a delivery device with crescent blade as described above may be used to form the intra-scleral pocket. Once the pocket is formed, the delivery device with the implant inside or mounted on the shaft is advanced through the pocket into the anterior chamber, whereupon the collector is released in the same manner as described above, resulting in its self-expansion. In this embodiment, the implant is loaded with the collector toward the distal-most end of the lumen or shaft of the delivery device and with the disperser toward the injector. Once the collector is ejected, the delivery device is withdrawn back to the pocket, during which time the connector is released. Then, when the distal end of the lumen or shaft of the delivery device has reached the pocket, the disperser is released in the pocket, resulting in the self-expansion of the disperser. Then the delivery device is withdrawn from the eye. The sclera is closed, and the conjunctiva is closed. Sutures may be used to keep the sclera and/or conjunctiva closed.

After any of the above-described procedures, further implants may be implanted. When it is desired to implant multiple implants, the delivery device can be loaded with multiple implants so that successive implants can be implanted without having to completely withdraw the delivery device.

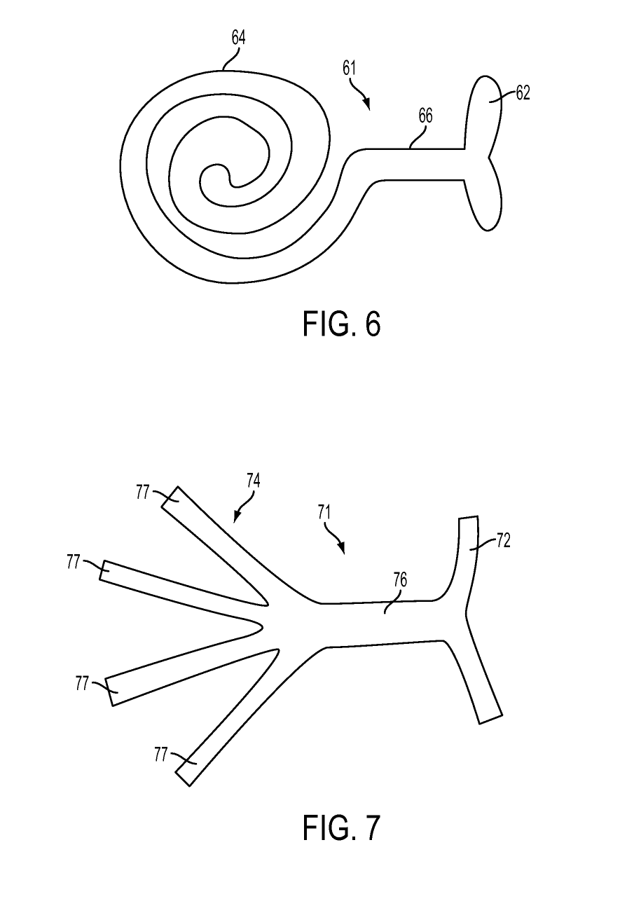

FIG. 6 shows an alternative implant design. The implant device 61 comprises an expandable fish-tail collector 62 and an expandable spiral disperser 64 connected by a connector 66. The implant device 61 is made of a flexible tube, as in the above embodiments, except that the tube is bifurcated to form the fish-tail collector 62. The tube has slits and/or holes as described above. The implant device 61 may be implanted using a delivery device as described above and in a similar manner as described above.

FIG. 7 shows another alternative implant design. The implant device 71 comprises an expandable fish-tail collector 72 and a disperser 74 in the form of an expandable set of fingers 77. The collector 72 and disperser 74 are connected by a connector 76. The implant device 71 is made of a flexible tube, as in the above embodiments, except that the tube is bifurcated to form the fish-tail collector 72 and branches out into a plurality of tubes to form the set of fingers 77. The tube has slits and/or holes as described above, and each finger 77 may be a tube with additional holes and/or slits. The multiple fingers 77 insure continuous flow should one of them become clogged. The implant device 71 may be implanted using a delivery device as described above and in a similar manner as described above. In one embodiment, the fingers 77 may be used to disperse the aqueous humor toward the collector vessels 10.

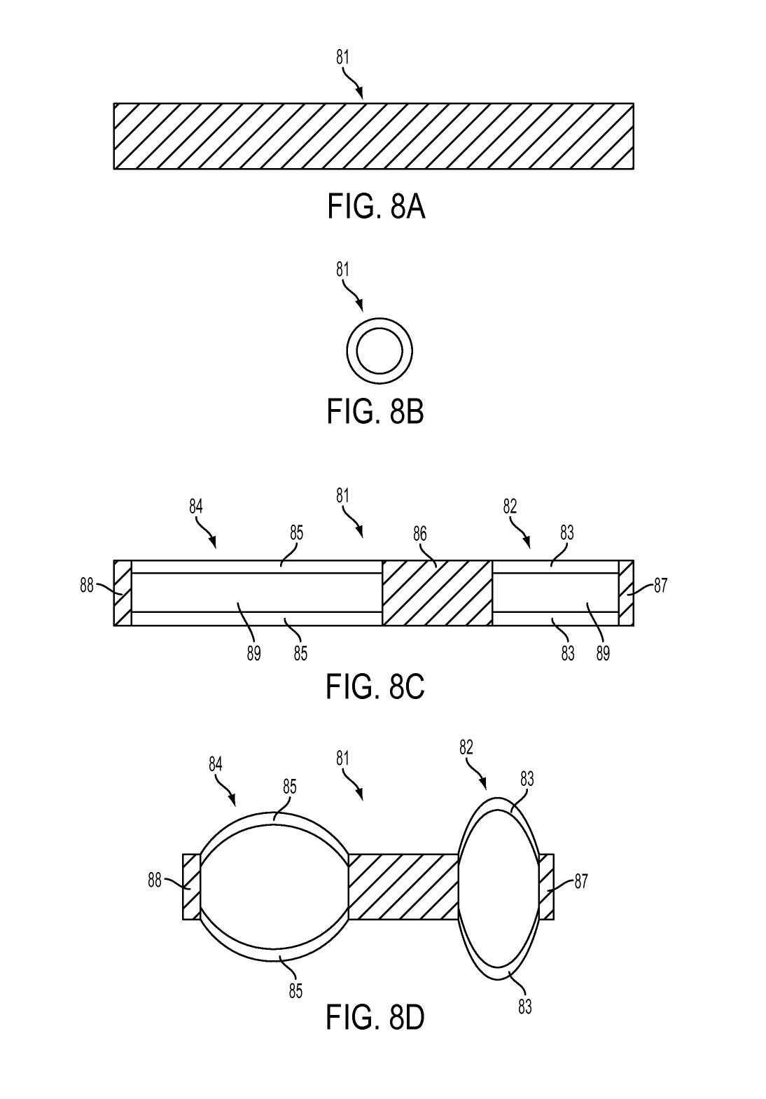

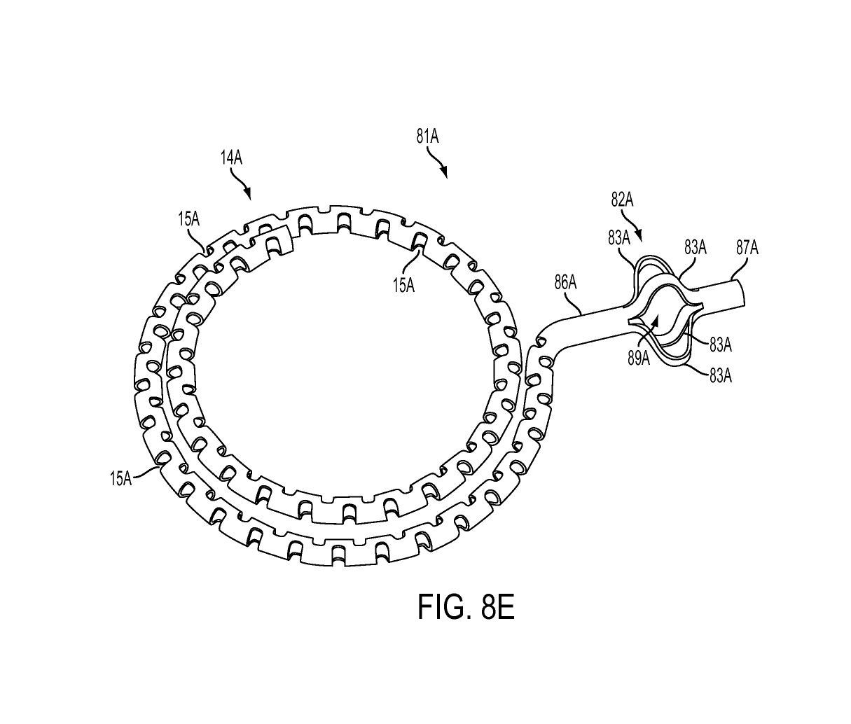

FIGS. 8A-8D show another alternative implant design. FIG. 8A shows a top view, FIG. 8B shows an end view, and FIG. 8C shows a side view of the implant device 81 in the constrained configuration. FIG. 8D shows a side view of the implant device 81 in the unconstrained, expanded configuration. The implant device 81 comprises a tube with cuts 89 in the side of the tube to form the collector and disperser. The cuts 89 can result in the collector and/or disperser having one, two, or more than two expandable side arms. In the illustrated embodiment, at one end, the cuts 89 result in two expandable side arms 83 forming the collector 82, and, at the other end, the cuts 89 result in two expandable side arms 85 forming the disperser 84. Uncut portions of the tube form the connector 86, an end 87 of the collector 82, and an end 88 of the disperser 84. The implant device 81 may be implanted using a delivery device as described above and in a similar manner as described above. In the constrained condition within the lumen of the delivery device, the expandable side arms 83, 85 are held in, so that the implant device 81 has a low profile generally as shown in FIGS. 8A-8C. When the implant device 81 is ejected from the lumen of the delivery device, the release of the implant device 81 from the constraint of the delivery device allows the expandable side arms 83, 85 to expand to their expanded condition, as shown in FIG. 8D. In this manner, the expanded collector 82 and disperser 84 perform similar functions as described above with respect to other implants.

FIG. 8E shows an alternative implant design comprising a collector similar to that in FIGS. 8A-8D and a disperser similar to that in FIGS. 1A-1B. FIG. 8E shows the implant device 81A in the unconstrained, expanded configuration. On one end, the implant device 81A comprises a tube with cuts 89A in the side of the tube to form the collector 82A. As indicted above with respect to FIGS. 8A-8D, the cuts 89A can result in the collector having one, two, or more than two expandable side arms. In the illustrated embodiment, the cuts 89A result in four expandable side arms 83A forming the collector 82A. Uncut portions of the tube form the connector 86A and an end 87A of the collector 82A. At the other end, the implant device 81A comprises a disperser 14A similar to that in FIGS. 1A-1B, having holes or slits 15A in the wall of the tube. The implant device 81A may be implanted using a delivery device as described above and in a similar manner as described above. In the constrained condition, the expandable side arms 83A are held in, and the disperser 14A is substantially straightened, so that the implant device 81A has a low profile. When the implant device 81A is ejected from the delivery device, the release of the implant device 81A from the constraint of the delivery device allows the expandable side arms 83A and the disperser 14A to expand to their expanded conditions. In this manner, the expanded collector 82A and disperser 14A perform similar functions as described above with respect to other implants. In other embodiments, the collector and disperser geometries may be reversed.

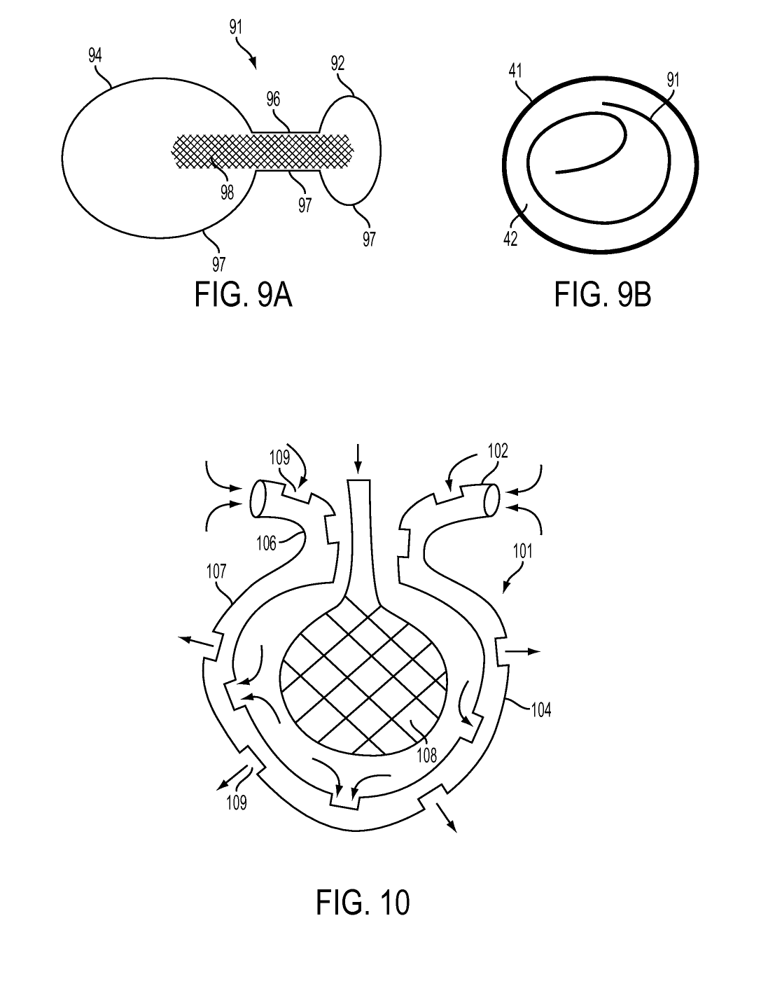

FIGS. 9A and 9B show another alternative implant design. FIG. 9A shows an implant device 91 comprising a collector 92 with an expandable frame and a disperser 94 with an expandable frame. The collector 92 and disperser 94 are connected by a connector 96. FIG. 9B shows the implant device 91 of FIG. 9A loaded inside a lumen 42 of shaft 41 of a delivery device 40. The frame 97 of the implant device 91 may be a wire or tube made of a suitable flexible material, such as nitinol or another material, as described above. Areas within the frame 97 may be coated or covered, in whole or in part, by a suitable coating or covering 98, for example a mesh of PVDF. The coating or covering can be made from biological or artificial material, can be degradable or stable, and can be made from solid or perforated material. In the area of the disperser 94, the covering 98 can help keep the pocket shape intact. When a mesh is used, the covering 98 provides additional flow paths through the mesh. The implant device 91 may be implanted using a delivery device as described above and in a similar manner as described above. For delivery, the flexible implant device 91 is rolled into a low profile and loaded into a lumen 42 of a delivery device. As the portions of the implant device 91 are ejected from the lumen 42, they unroll to their unconstrained configuration as shown in FIG. 9A.

FIG. 10 shows another version of an implant device 101 comprising a collector 102, a connector 106, and a disperser 104 having an expandable frame. The frame 107 in this example is a tube made of a suitable flexible material as described above. Holes or slits 109 are formed in the tube. Areas within the frame 107 may be coated or covered, in whole or in part, by a suitable coating or covering 108, as described above. In the area of the disperser 104, the covering 108 can help keep the pocket shape intact. When a mesh is used, the covering 108 provides additional flow paths through the mesh. The implant device 101 may be implanted using a delivery device as described above and in a similar manner as described above. The implant device 101 may be rolled or compressed for loading into the lumen of a delivery device.

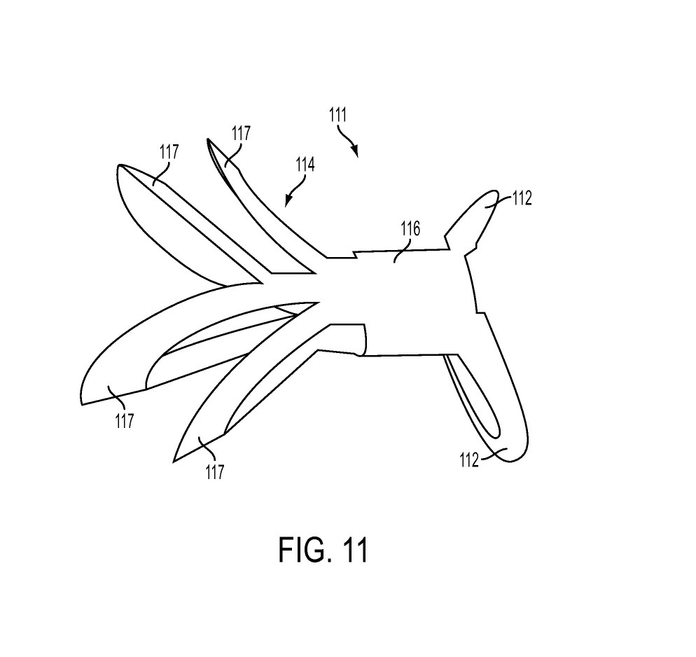

FIG. 11 shows another alternative implant design. The implant device 111 comprises an expandable fish-tail collector 112 and a disperser 114 in the form of an expandable set of fingers 117. The collector 112 and disperser 114 are connected by a connector 116. The implant device 111 is made of a flexible tube, as in the above embodiments, except that the tube is bifurcated to form the fish-tail collector 112 and branches out into a plurality of tube parts to form the set of fingers 117. The tube may have one or more slits and/or holes as described above, and each finger 117 may be a tube with additional holes and/or slits. The multiple fingers 117 may be cut and shaped from the same original tube as that used to form the connector 116 and collector 112. The multiple fingers 117 insure continuous flow should one or more of them become clogged. The implant device 111 may be implanted using a delivery device as described above and in a similar manner as described above. In one embodiment, the fingers 117 may be used to disperse the aqueous humor toward the collector vessels 10.

Other variations of the above described implants are possible. The expandable collector and/or disperser may be in the shape of a spiral, a conical helix, a fish tail, a set of fingers, a set of arms, a frame, a dish, or any other suitable expandable shape. A conical helix, as opposed to a flat spiral, when used as the disperser, can help separate the top and bottom of the pocket and provide a large reservoir that is less susceptible to becoming closed. Other shapes (e.g., extending fingers, conical shapes, etc.) also can serve this purpose. Any of these shapes may be provided with slits and/or holes to improve collection of fluid and reduce the chance of obstruction. Different sizes of slits and/or holes may be used. In some instances, it may be desired that the disperser have only small openings or no openings close to the connector, in order to divert the flow of fluid far from the connector. Anchors such as barbs or hooks may be provided on the implant to prevent migration.

In some embodiments, only one of the disperser or collector may be expandable. For example, for use with an ab-externo implantation procedure, the collector may be expandable, while the disperser may be rigid or semi-rigid. In other embodiments, an implant having a configuration generally as described herein may be rigid or semi-rigid.

The implant device may be provided with features for controlling fluid flow. For example, when a tube is used, the inside of the tube may be provided with an impediment that provides resistance to reduce flow rate. Alternatively, a valve may be positioned in the tube for regulating fluid flow. The valve may open and close, and let in more or less flow, based on pressure fluctuation or based on control by an actuator. Additionally, actuators may open and close holes in the implant, for example in the disperser, to increase or decrease the flow in certain areas.

An implant device as described herein may also incorporate other features. For example, it may carry a pressure sensor, flow sensor or flow meter.

Based on the above description and the accompanying drawings, the principles and operation of the invention, as well as how to make and use the invention, can be understood by persons of ordinary skill in the art. Many embodiments and variations are possible that take advantage of the principles and operation of the invention described herein. The examples described herein and shown in the accompanying drawings are meant as examples only and are not intended to be limiting of the scope of the invention defined by the appended claims.

What is claimed is:

1. An implant for regulating the flow of fluid, wherein the implant device comprises:
a collector; and
a disperser;
wherein at least one of the collector and the disperser is self-expandable from a constrained low-profile configuration to an unconstrained expanded configuration.

2. An implant as recited in claim 1, wherein the collector and disperser are connected by a connector.

3. An implant as recited in claim 1, wherein the collector has a generally spiral or conical-helical shape.

4. An implant as recited in claim 1, wherein the disperser has a generally spiral or conical-helical shape.

5. An implant as recited in claim 1, wherein both the collector and the disperser are self-expandable from a constrained low-profile configuration to an unconstrained expanded configuration.

6. An implant as recited in claim 1, wherein the implant is comprised, at least in part, of nitinol.

7. An implant as recited in claim 1, wherein at least one of the collector and the disperser has an expandable shape selected from the group of: spiral, helical, conical-helical, fish-tail, a set of fingers, a set of side arms, an expandable frame, and an expandable dish.

8. A delivery device for delivering an implant for regulating the flow of fluid, wherein at least a part of the implant is self-expandable, such that the implant has a constrained low-profile configuration and an unconstrained expanded configuration;
wherein the delivery device comprises a shaft adapted to hold the implant in its low-profile configuration during delivery of the implant.

9. A delivery device as recited in claim 8, wherein the delivery device further comprises an injector capable of ejecting the implant from the delivery device.

10. A delivery device as recited in claim 8, wherein the delivery device further comprises a cutting edge.

11. A delivery device as recited in claim 8, wherein the delivery device further comprises an illuminator.

12. A method of implanting an implant for regulating the flow of fluid, wherein the implant comprises a collector and a disperser and wherein at least one of the collector and the disperser is self-expandable from a constrained low-profile configuration to an unconstrained expanded configuration, wherein the method comprises:
advancing a delivery device to a desired implantation site, wherein the delivery device comprises a shaft, and wherein the shaft holds the implant in a low-profile configuration; and
ejecting the implant from the shaft of the delivery device, whereby the implant self-expands from a constrained low-profile configuration to an unconstrained expanded configuration.

13. A method as recited in claim 12, further comprising:
forming a pocket within the sclera of an eye;
wherein the step of ejecting the implant from the shaft of the delivery device comprises ejecting the disperser of the implant into the pocket, whereby the disperser self-expands to an unconstrained expanded configuration in the pocket.

14. A method as recited in claim 13, wherein the step of ejecting the implant from the shaft of the delivery device further comprises withdrawing the delivery device from the pocket and ejecting the collector of the implant into the anterior chamber of the eye, whereby the collector self-expands to an unconstrained expanded configuration in the anterior chamber.

15. A method as recited in claim 12, wherein the step of ejecting the implant from the shaft of the delivery device comprises ejecting the collector of the implant into the anterior chamber of the eye, whereby the collector self-expands to an unconstrained expanded configuration in the anterior chamber.

16. A method as recited in claim 12, wherein the collector and the disperser are connected by a connector.

17. A method as recited in claim 12, wherein the collector has a generally spiral or conical-helical shape.

18. A method as recited in claim 12, wherein the disperser has a generally spiral or conical-helical shape.

19. A method as recited in claim 12, wherein the method is performed ab-interno.

20. A method as recited in claim 12, wherein the method is performed ab-externo.
